(19) United States
(12) Patent Application Publication
(10) Pub. No.: US 2023/0404455 A1
(43) Pub. Date: Dec. 21, 2023

(54) PLATFORM FOR A DIGITAL PSYCHOLOGICAL ASSESSMENT TOOL

(71) Applicant: JLY holdings, LLC, Rochester Hills, MI (US)

(72) Inventors: Joel Young, Bloomfield Hills, MI (US); Margot Nash, Chicago, IL (US)

(21) Appl. No.: 18/211,506

(22) Filed: Jun. 19, 2023

Related U.S. Application Data
(60) Provisional application No. 63/353,190, filed on Jun. 17, 2022.

(51) Int. Cl. A61B 5/16; G09B 7/00

(52) U.S. Cl. CPC A61B 5/165; G09B 7/00

(57) ABSTRACT

A computerized assessment tool and a platform for a psychological assessment tool that provides screening tests, confirmatory tests, or questionnaires to a user and receives input from the user via a display device; assesses information gained from the screening tests, confirmatory tests, or questionnaires against a set of condition-specific rating scales to determine diagnostic specifiers that are indicative of existence, non-existence, or likelihood of one or more mental health conditions; and generates a report based on the diagnostic specifiers.

### CROSS REFERENCE TO RELATED APPLICATIONS

This application claims priority to U.S. Provisional Patent Application No. 63/353,190, filed on Jun. 17, 2022, entitled “Platform for a Digital Psychological Assessment Tool,” which is incorporated herein by reference in its entirety.

### TECHNICAL FIELD

The present disclosure relates generally to computerized psychological assessment tools and, more specifically, a platform for a psychological assessment tool that uses an innovative approach to broad-scope assessment and statistical approach to help users accurately interpret results.

### BACKGROUND

Currently, there is a shortage of clinicians that are qualified to diagnose and treat those suffering with mental health concerns requiring medication. As a result, those individuals routinely wait for months or years before getting in for an appointment with a psychiatrist, see their primary care practitioner who may not be equipped to make a proper diagnosis, or sign up for a subscription psychiatry network. However, these courses of action may be detrimental to their well-being.

Moreover, even if a patient is able to see a clinician that clinician may not have the time or the training to assess the patient, which can result in things like misdiagnoses, delayed diagnoses or the like. Further still, current assessment tools can be difficult to use, are not consolidated in an efficient manner and are often not effective. Therefore, there is a need for a psychological assessment with a high degree of comprehensiveness and depth.

### SUMMARY

The following presents a summary of this disclosure to provide a basic understanding of some aspects. This summary is intended to neither identify key or critical elements nor define any limitations of embodiments or claims. This summary may provide a simplified overview of some aspects that may be described in greater detail in other portions of this disclosure. Furthermore, any of the described aspects may be isolated or combined with other described aspects without limitation to the same effect as if they had been described separately in every possible combination explicitly.

A platform is described herein that provides a digital psychiatric assessment that provides one or more reports. The platform uses a tiered process that combines a public health approach to universal screening with an individualized, hypothesis-testing approach of psychological and psychiatric evaluations. The platform uses a rigorous statistical approach that allows it to obtain results with a higher degree of confidence than a single rating scale for a given condition. The platform screens individuals for symptoms associated with various psychiatric conditions. The platform directs individuals to complete additional targeted, norm-referenced or criterion-referenced rating tests pertaining to the psychiatric conditions that were elevated on the screening. The platform directs individuals to complete a questionnaire to identify demographic information and other life factors that may contribute to mental health. The platform assesses the information gained against sets of condition-specific rating scales to generate a report that provides suggestions about indicated diagnostic specifiers and an individualized guide to facilitate the individual taking the next steps. While the report does not diagnose any specific condition, it provides information to facilitate the individual more readily receiving an accurate diagnosis from a health care clinician.

In one aspect, a platform to provide digital assessments of mental health comprises: a screening engine that provides screening tests, confirmatory tests, or questionnaires to a user and receives input from the user via a display device; a testing engine that assesses information gained from the screening tests, confirmatory tests, or questionnaires against a set of condition-specific rating scales to determine diagnostic specifiers that are indicative of existence, non-existence, or likelihood of one or more mental health conditions; and a report generator that generates a report based on the diagnostic specifiers.

In another aspect, the platform further comprises a profile generator that generates a profile for the user based on a user's input upon registering on a website and providing characteristics such as, but not limited to, name, email, and age.

In another aspect, the screening engine screens the user for symptoms associated with psychiatric conditions through a series of questions based on a Diagnostic and Statistical Manual of Mental Disorders, Fifth Edition (DSM-5).

In another aspect, the report is personalized based on a user's response to life factor questions that are further provided by the screening engine.

In another aspect, a system comprises: a host computing device that includes a web server and an application server; and a user device in communication with the host computing device by way of a communication framework to provide a user access to an assessment platform via the web server and the application server; wherein the assessment platform comprises: a screening engine that provides screening tests, confirmatory tests, or questionnaires to a user and receives input from the user via a display device; a testing engine that assesses information gained from the screening tests, confirmatory tests, or questionnaires against a set of condition-specific rating scales to determine diagnostic specifiers that are indicative of existence, non-existence, or likelihood of one or more mental health conditions; and a report generator that generates a report based on the diagnostic specifiers.

In another aspect, the assessment platform further comprises a profile generator that generates a profile for the user based on a user's input upon registering on a website and providing characteristics such as, but not limited to, name, email, and age.

In another aspect, the screening engine, of the system, screens the user for symptoms associated with psychiatric conditions through a series of questions based on a Diagnostic and Statistical Manual of Mental Disorders, Fifth Edition (DSM-5).

In another aspect, the assessment platform, of the system, via the testing engine, determines that one or more mental health conditions have been supported or not when a user's pattern of responses on individual tests resulted in a majority (considering the published sensitivity/specificity of each test in the set) of positive versus negative scores for a specific condition.

In another aspect, the report comprises a first part that includes a comprehensive psychoeducation and a second part that includes information a healthcare provider would need in order to make sound decisions about a user's mental health.

In another aspect, the second part comprises: (a) demographic information, (b) reason for testing, (c) relevant medical history, (d) evaluation of suicide risk, (e) data on possible conditions detected, (f) raw or underlying data supporting possible conditions detected, or (g) drug tables for relevant conditions.

In another aspect, a method comprises: administering screening tests, confirmatory tests, or questionnaires to a user and receiving input from the user via a user device; assessing information gained from the screening tests, confirmatory tests, or questionnaires against sets of condition-specific rating scales to determine diagnostic requirements that are indicative of an existence, non-existence, or likelihood of a mental health condition; and generating a report based on the diagnostic requirements.

In another aspect, the screening tests prompt the user to answer a checklist of common sign and symptoms of more than 50 psychiatric conditions to provide a list of hypothesized conditions for further testing.

In another aspect, the checklist of common sign and symptoms has a one-to-one alignment with symptoms enumerated in a Diagnostic and Statistical Manual of Mental Disorders, Fifth Edition (DSM-5), and the checklist of common sign and symptoms prompts the user to endorse whether the symptoms for a given condition is presence or absence.

In another aspect, the screening tests comprises three tiers, and wherein: Tier 1 comprises questions regarding broadly described symptoms that are required per the DSM-5 for certain categories of DSM-5 disorders; Tier 2 comprises questions regarding symptoms whose presence is required for certain DSM-5 disorders to be present; and Tier 3 comprises questions regarding the symptoms of which the presence of a certain amount is required for the certain DSM-5 disorders to be present.

In another aspect, the method further comprises administering a set of one or more daily impact questionnaires that comprises questions about how symptoms impact a user's daily life.

In another aspect, if, based on a user's characteristics and symptom endorsements the presence of one or more DSM-5 disorders is possible, the method further comprises administering a set of one to four established and well -known self-report survey-type rating scales validated and peer-reviewed for use in screening for possible disorders.

In another aspect, whether the presence of one or more DSM-5 disorders is possible means that disorder-specific Tier 3 symptoms were endorsed and at least one less than a DSMS-required volume of the disorder-specific Tier 3 symptoms were endorsed.

In another aspect, the method further comprises administering a set of one or more life factor questionnaires that comprises questions about a user's life that may help a healthcare provider tailor treatments for the user.

In another aspect, based on sensitivity and specificity data of the sets of condition-specific rating scales, a probability level of a true positive finding is calculated by a formula: 1 [maximum possible probability]−(Error % test1×Error % test2× . . . Error % test(n)), wherein a conservative estimate of probability is used by assigning an assumed error rate to a measure through setting an error value equal to: (1−Sensitivity)+(1−Specificity). This conservative estimate of probability may be presented as a % for each condition or as a level of evidence such as strong, moderate, or mild

In yet another aspect, the method further comprises displaying the report on a user device.

The following description and the drawings disclose various illustrative aspects. Some improvements and novel aspects may be expressly identified, while others may be apparent from the description and drawings.

### BRIEF DESCRIPTION OF THE DRAWINGS

Operation of the present disclosure may be better understood by reference to the following detailed description taken in connection with the following illustrations, wherein:

FIG. 1 is a block diagram of a platform to host a digital psychological assessment tool, in accordance with the embodiments disclosed herein.

FIG. 2 is an example flowchart of a method for a platform to provide a mental health diagnostic specifier report in accordance with the embodiments disclosed herein.

FIG. 3 is a schematic diagram of embodiments of a system for furnishing an online psychological assessment in accordance with the embodiments disclosed herein.

FIG. 4 is a block diagram of a platform to host a digital psychological assessment tool including a profile generator and a user interface engine, in accordance with the embodiments disclosed herein.

FIG. 5 is an example flowchart of a method, in accordance with the embodiments disclosed herein.

FIGS. 6-18 illustrate an example user journey, in accordance with the embodiments disclosed herein.

### DETAILED DESCRIPTION

Reference will now be made in detail to exemplary embodiments of the present disclosure, examples of which are illustrated in the accompanying drawings. It is to be understood that other embodiments may be utilized and structural and functional changes may be made. Moreover, features of the various embodiments may be combined or altered. As such, the following description is presented by way of illustration only and should not limit in any way the various alternatives and modifications that may be made to the illustrated embodiments. It should be understood that aspects of this disclosure may be practiced with other embodiments not necessarily including all aspects described herein, etc.

As used herein, the words “example” and “exemplary” mean an instance, or illustration. The words “example” or “exemplary” do not indicate a key or preferred aspect or embodiment. The word “or” is intended to be inclusive rather than exclusive, unless context suggests otherwise. As an example, the phrase “A employs B or C,” includes any inclusive permutation (e.g., A employs B; A employs C; or A employs both B and C). As another matter, the articles “a” and “an” are generally intended to mean “one or more” unless context suggests otherwise.

A “processor”, as used herein, processes signals and performs general computing and arithmetic functions. Signals processed by the processor can include digital signals, data signals, computer instructions, processor instructions, messages, a bit, a bit stream, or other means that can be received, transmitted and/or detected. Generally, the processor can be a variety of various processors including multiple single and multicore processors and co-processors and other multiple single and multicore processor and co-processor architectures. The processor can include various modules to execute various functions.

A “memory”, as used herein can include volatile memory and/or nonvolatile memory. Non-volatile memory can include, for example, ROM (read only memory), PROM (programmable read only memory), EPROM (erasable PROM), and EEPROM (electrically erasable PROM). Volatile memory can include, for example, RAM (random access memory), synchronous RAM (SRAM), dynamic RAM (DRAM), synchronous DRAM (SDRAM), double data rate SDRAM (DDRSDRAM), and direct RAM bus RAM (DRRAM). The memory can also include a disk. The memory can store an operating system that controls or allocates resources of a computing device. The memory can also store data for use by the processor.

A “disk”, as used herein can be, for example, a magnetic disk drive, a solid-state disk drive, a floppy disk drive, a tape drive, a Zip drive, a flash memory card, and/or a memory stick. Furthermore, the disk can be a CD-ROM (compact disk ROM), a CD recordable drive (CD-R drive), a CD rewritable drive (CD-RW drive), and/or a digital video ROM drive (DVD ROM). The disk can store an operating system and/or program that controls or allocates resources of a computing device.

Some portions of the detailed description that follows are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps (instructions) leading to a desired result. The steps are those requiring physical manipulations of physical quantities. Usually, though not necessarily, these quantities take the form of electrical, magnetic or optical non-transitory signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations or transformation of physical quantities or representations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or “determining” or the like, refer to the action and processes of a computer system, or similar electronic computing device (such as a specific computing machine), that manipulates and transforms data represented as physical (electronic) quantities within the computer system memories or registers or other such information storage, transmission or display devices.

Certain aspects of the embodiments described herein include process steps and instructions described herein in the form of an algorithm. It should be noted that the process steps and instructions of the embodiments could be embodied in software, firmware or hardware, and when embodied in software, could be downloaded to reside on and be operated from different platforms used by a variety of operating systems. The embodiments can also be in a computer program product that can be executed on a computing system.

The embodiments also relates to an apparatus for performing the operations herein. This apparatus can be specially constructed for the purposes, e.g., a specific computer, or it can comprise a general-purpose computer selectively activated or reconfigured by a computer program stored in the computer. Such a computer program can be stored in a non-transitory computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each electrically connected to a computer system bus. Furthermore, the computers referred to in the specification can include a single processor or can be architectures employing multiple processor designs for increased computing capability.

The algorithms and displays presented herein are not inherently related to any particular computer or other apparatus. Various general-purpose systems can also be used with programs in accordance with the teachings herein, or it can prove convenient to construct more specialized apparatus to perform the method steps. The structure for a variety of these systems will appear from the description below. In addition, the embodiments are not described with reference to any particular programming language. It will be appreciated that a variety of programming languages can be used to implement the teachings of the embodiments as described herein, and any references below to specific languages are provided for disclosure of enablement and best mode of the embodiments.

In addition, the language used in the specification has been principally selected for readability and instructional purposes, and may not have been selected to delineate or circumscribe the inventive subject matter. Accordingly, the disclosure of the embodiments is intended to be illustrative, but not limiting, of the scope of the embodiments, which is set forth in the claims.

Mental health conditions are routinely treated in the absence of quantifiable data, often to the detriment of the individual. The use of data has proven benefit, yet its collection is not a regular practice, even in psychiatric settings. Accurate diagnosis and treatment of psychological conditions requires a lengthy process of asking about a wide variety of symptoms, each with a high degree of precision. Due to supply and demand constraints, primary care physicians, who do not have the time or training to accurately do so, frequently diagnose and provide medical treatment for mental health concerns, more than psychiatrists. As a result of specialist shortages, over 60% of psychiatric medications are prescribed by primary care providers. Patients often endure ineffective treatment or no treatment as a result. Research has shown rates of misdiagnosis in the primary care setting ranging from approximately 65% to 98% for even the most common mental health conditions. Vermani, Monica et al. “Rates of detection of mood and anxiety disorders in primary care: a descriptive, cross-sectional study.” The primary care companion for CNS disorders vol. 13,2 (2011): PCC.10m01013. doi:10.4088/PCC.10m01013. Thus, the risks of self-diagnosis and inaccurate clinical decisions are rampant and in need of public health-promoting products. Even for people with conditions that are more like to visit their doctor, such as anxiety disorder, their symptoms are often attributed to physical causes. Id. Another example is depression because the core symptoms of depression is fatigue, decreased energy, and lack of motivation, which are often misinterpreted by primary care physicians as the result of physical causes. Id. The misdiagnosed or undiagnosed disorders results in self-medication, substance abuse, increased doctor's visits, inappropriate treatment, social isolation, etc. Id. The diagnostic tools that are available are not being used by physicians because they are lengthy, time consuming, and must be administered by trained clinicians. Id.

The embodiments disclosed herein offers individuals and practitioners a way to assess a broad range of psychological symptoms and conditions with expert precision through an online tool or platform enabling either user (e.g., individuals or practitioners) to take accurate next steps. The embodiments disclosed herein educate and empower adults with symptom awareness and curated information, encourage individuals to seek care from qualified professionals, and equip individuals with a comprehensive report for productive conversations with providers.

It will become evident that the systems and methods disclosed herein is sufficient and does not require a patient to go to multiple specialists/clinicians. The mental health assessments can be done in a single sitting in front of a single computer. The systems and methods disclosed herein is comprehensive, and the mental health assessments can get done in a short time what could otherwise take multiple visits to a clinician. The faster a user/patient receives the results the faster the user/patient can receive treatment. This may help improve the life of the patient quicker. The systems and methods disclosed herein is also comprehensive in that it can diagnose/predict more conditions than a clinician. The systems and methods disclosed herein can also be more accurate in that it can remove a clinician's bias that may otherwise be there. This is an important distinction between a computer and a person. This may be particularly useful if a patient has multiple conditions. A clinician may diagnose a single condition believing the issues were addressed. If the patient is suffering from multiple conditions, the present systems and methods disclosed herein can provide results to all of them because it is not biased. The systems and methods disclosed herein is updatable. New understandings and discoveries can be updated into the systems and methods disclosed herein. This makes it far more efficient as a clinician would have to otherwise research the issues on his/her own or would need to take classes. This can make the system more accurate and more efficient. The systems and methods disclosed herein is scalable. It can provide results to an immeasurable amount of patients where a single clinician can only do that for a select few patients. This will allow more patients to ultimately get their results and get treated. Obviously the more patients that get treated, the better off society is.

Provided is a web-based mental health assessment and recommendations software (although the present disclosure contemplates downloadable software, software as a service and/or an app) with a comprehensive questionnaire that promotes shared decision-making and clear disclaimers. The comprehensive questionnaire comprises a preliminary symptom checklist and validated psychiatric screening scales selected based on data from peer-reviewed sources to facilitate collaboration between users and healthcare providers by emphasizing the tool's non-diagnostic nature and the importance of professional involvement, which encourages users to share reports with their healthcare providers.

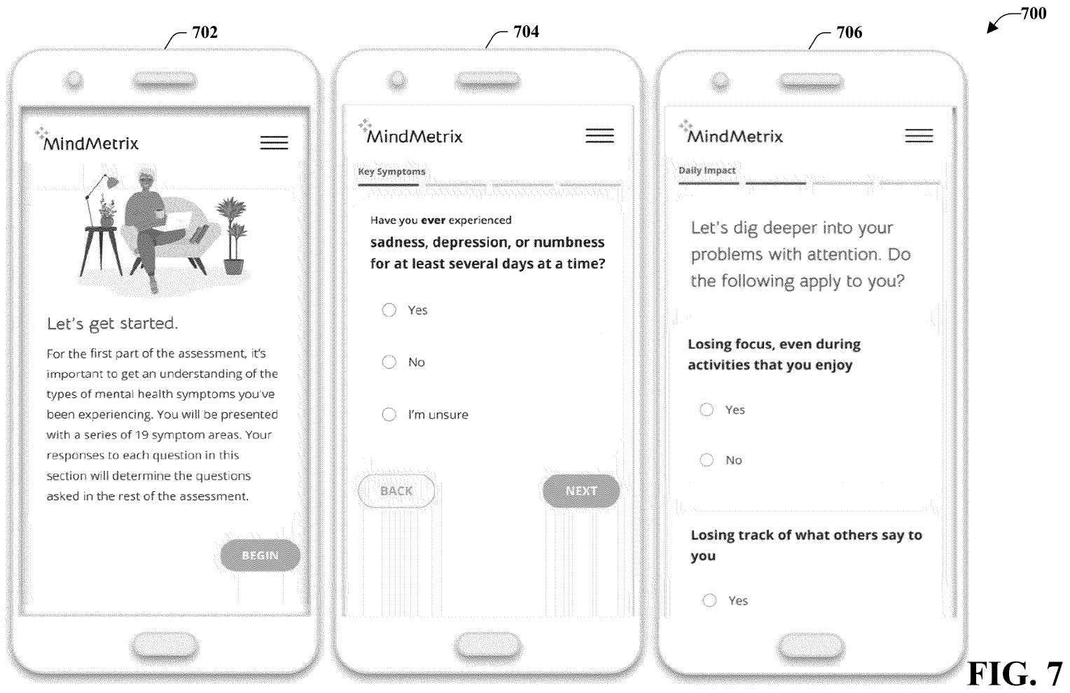

As described below, a platform hosting a mental health assessment tool includes a user interface (such as that shown in FIG. 7) designed for test-takers to undergo a psychiatric screening and assessment without the assistance of a clinician. A diagnostic screening portion of the assessment process covers symptoms of more than 50 mental health conditions using tests based on the Diagnostic and Statistical Manual of Mental Disorders, Fifth Edition (DSM-5) criteria required for diagnosis. The screening results generate a number of “hypotheses” to test using condition-specific test-sets. The condition-specific tests may be of any appropriate type and the present teachings are not limited to specific tests.

The condition-specific test-sets use a statistical approach to obtain results with a high degree of confidence. Specifically, each of the conditions has a corresponding set of condition-specific rating scales (e.g., ADHD-specific (attention-deficit/hyperactivity disorder) rating scales, Generalized Anxiety-specific rating scales, Depression-specific rating scales, etc.). The scales are based on previously published sensitivity (likelihood of a “true positive”) and specificity (likelihood of a “true negative”) for a condition at a designated clinical cut-off score. Individual rating scales or psychometric tests are selected to function within a set of tests. The platform uses the published sensitivity/specificity statistics of each test to weight its result in context of the other tests in the set. In such a manner, no single measurement is relied upon solely to arrive at an indicated possible or likely condition as part of the assessment. The platform generates impressions of which conditions are present or are likely present based not only on the elevated assessment results of the condition, but also by ruling out the DSM-5's required “related” or “similar” conditions. The platform performs a differential diagnosis process based on its broad scope and reports the information collected and the information that still needs to be assessed if the system was not able to determine a result for a given related condition. After the assessment, the platform provides (i) information written by clinicians that is relevant to their “elevated” conditions and (ii) a report that presents the data to a practitioner of any kind that would need to be able to make an accurate decision, including a medication table that is practical and concise for relevant conditions. The report may be a clinician-oriented document that reports results of the multiple mental health conditions assessed by the platform. For each condition, the report displays test-taker screening results, assessment results with likelihood levels, differential diagnoses, and recommendations for areas of further evaluation.

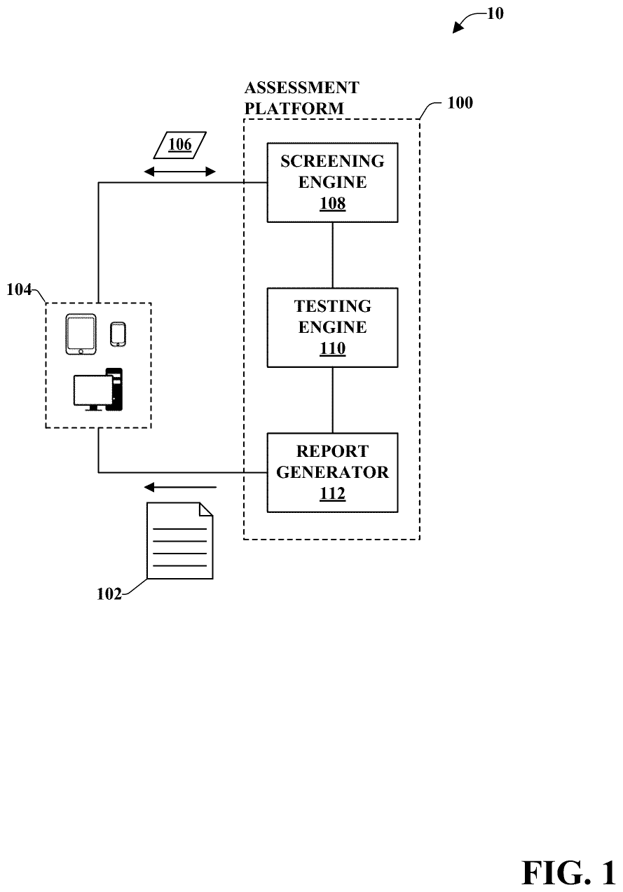

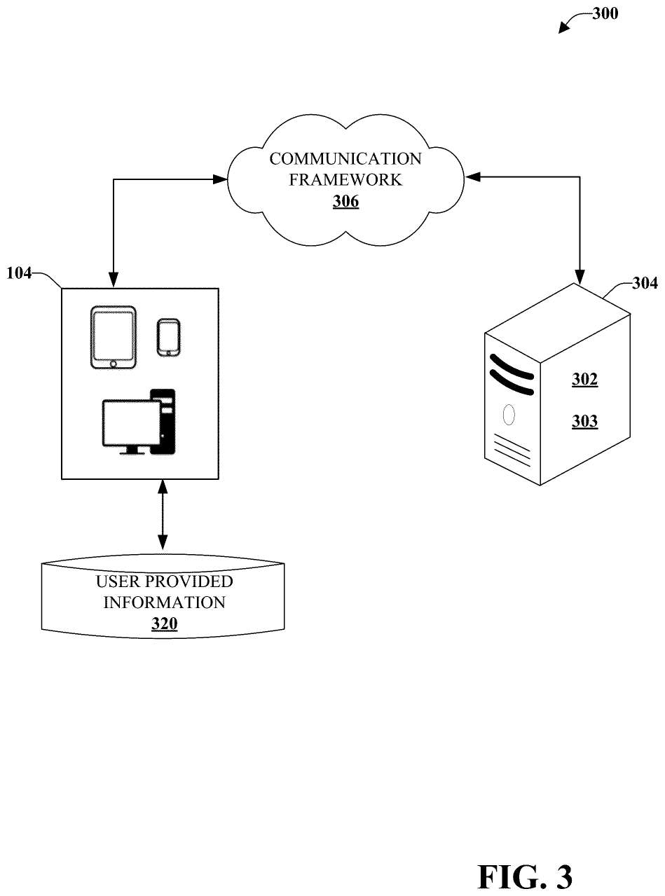

FIG. 1 illustrates an example system 10 comprising a system architecture 300 (e.g., see FIG. 3) and an example platform 100 that may be a web application (e.g., psychological assessment web application) that hosts a digital psychological assessment tool to provide a report 102 to an individual that interacts with the platform 100. In the illustrated example, an individual uses a computing device 104 (e.g., user device, display device, etc.) such as, but not limited to, a desktop computer, a tablet computer, a laptop computer, a smartphone, etc., to interact with the platform 100 (e.g., via a software application such as a web browser). The platform 100 exchanges data 106 with the computing device 104 as described herein.

In the illustrated examples, the platform 100 includes a screening engine 108, a testing engine 110, and a report generator 112. The screening engine 108 provides screening tests (e.g., a multiple-tiered process), confirmatory tests (e.g., targeted tests based on the screening, individual rating scales, psychometric tests, etc.), and questionnaires (e.g., life factor questionnaires) to the individuals to the computing device 104 and receives input from the individual via the computing device 104. The testing engine 110 assesses the information gained from screening tests, confirmatory tests, and questionnaires against sets of condition-specific rating scales to determine diagnostic specifiers (e.g., requirements) that are indicative of the existence or non-existence of a mental health condition, conditions or likelihood of condition(s). The report generator 112, based on the diagnostic specifiers (e.g., requirements), generates the report 102 to be provided to the user via the computing device 104.

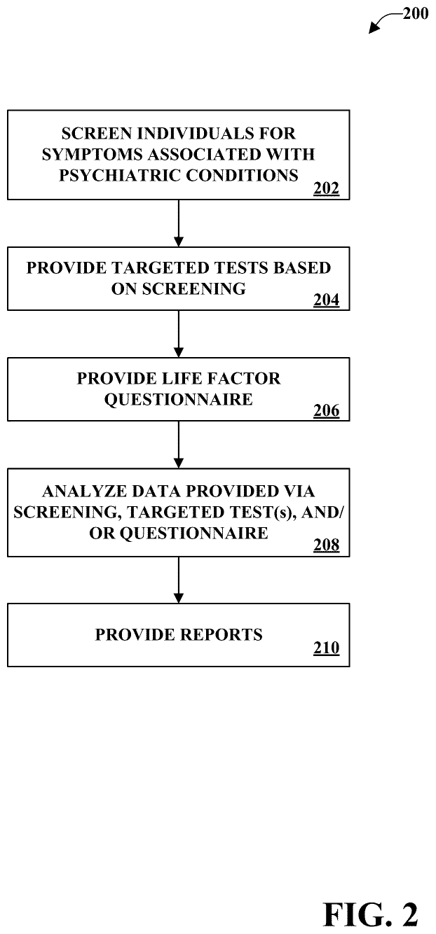

FIG. 2 is an example flowchart of a method 200 for the assessment platform 100 (e.g., generally, platform 100) to provide a mental health diagnostic specified report 102. The platform 100 screens the individual for symptoms associated with psychiatric conditions (block 202). The platform 100, via the screening engine 108, screens the individual for symptoms associated with psychiatric conditions through a series of questions (via a user interface) based on, for example, the DSM-5 (or any subsequent revisions and/or editions). While the example herein are described using the DSM-5, any other diagnostic manual or methodology that specifies diagnostic criteria may be used. The screening presents a thorough questionnaire that adapts to include relevant symptoms as the test-taker answers questions that tightly align with DSM-5 criteria. That is, as the user answers questions, the platform constructs the remaining screening questionnaire based on the answer previously given. In some examples, the screening includes fifty conditions (although any appropriate number may be used) encompassing many of the most common psychiatric conditions present in the adult population. For example, the screening may include questions relevant to neurodevelopmental disorders (e.g., attention deficit/hyperactivity disorder (ADHD), autism spectrum disorder, etc.), anxiety disorders (e.g., generalized anxiety disorder, social anxiety disorder, panic disorder, agoraphobia, specific phobias, etc.), obsessive-compulsive and related disorders (e.g., obsessive-compulsive disorder, body dysmorphic disorder, hoarding disorder, trichotillomania, excoriation disorder, etc.), trauma- and stressor-related disorders (e.g., post-traumatic stress disorder, acute stress disorder, etc.), somatic symptom and related disorders (e.g., somatic symptoms disorder, illness anxiety disorder, fibromyalgia, chronic fatigue, etc.), depressive disorders (e.g., major depressive disorder, major depressive disorder with peripartum onset, disruptive mood dysregulation disorder, etc.), bipolar and related disorders (e.g., bipolar I, bipolar II, cyclothymic disorder, etc.), feeding and eating disorders (e.g., anorexia nervosa—restricting type, anorexia nervosa—binge/purge type, avoidant/restrictive food intake disorder, bulimia nervosa, binge-eating disorder, etc.), schizophrenia spectrum and other psychotic disorders (e.g., schizophrenia, etc.), personality disorders (e.g., borderline personality disorder, etc.), personality traits (e.g., narcissistic personality traits, antisocial personality traits, etc.), substance-related and addictive disorders (e.g., alcohol use disorder, other substance use disorders, etc.), sleep-wake disorders (e.g., insomnia, narcolepsy, etc.), and/or gender dysphoria, etc. The assessment platform 200 may comprise any set of the foregoing in any order and combination. Moreover, the assessment platform 200 ma be configured to have any conditions added, updated or modified. The assessment platform 200 may comprise any number of conditions and not just the fifty identified.

The screening questionnaire includes questions related to multiple psychiatric conditions to ensure important potentially co-occurring symptoms or conditions are not overlooked. The screening engine 108 generates a list of hypothesized present diagnoses, based on engagement by the user with questions constructed for criterion and face validity with one-to-one alignment with DSM-5 diagnostic criteria. The hypotheses created by the questions are not as findings or diagnosis. Rather, the screening engine 108 uses the hypotheses to select which sets of validated measures to administer (e.g., in the next step). A hypothesis formed by the questionnaire is only treated as valid if it is confirmed through a set of psychometrics (e.g., in the next steps). In this way, the psychometric findings of the validated measures are triangulated with self-reported presence of DSM-5 diagnostic criteria by the platform.

The platform 100, via the screening engine 108, provides one or more self-report tests based on the screening (e.g., confirmatory tests, targeted tests based on the screening, individual rating scales, psychometric tests, etc.) to validate hypotheses generated through screening (block 204). The screening engine 108 provides the test(s) for the users to complete that are targeted, norm-referenced and/or criterion-referenced rating scales pertaining to the psychiatric conditions that were elevated during the screening. The criterion-referenced assessment tool measures the user's symptoms or functioning against a predetermined criteria or standards. In most examples, the screening engine 108 uses “cut-off scores” that have been empirically-derived to sort the user into “low-risk” and “high-risk” categories for a mental health condition. The norm-referenced assessment tools compare the user's symptoms or functioning to that of a normative comparison group. Although the characteristics of the normative group vary depending on the assessment, they are generally a nationally representative sample of several thousand individuals. Percentile ranks and standard scores that indicate one's symptoms or functioning falls in the “above average,” “average,” or “low average” range compared to their peers in the norming sample, are examples of norm-referenced scores.

Each of the conditions assessed by the testing engine 110 has a corresponding set of condition-specific rating scales (e.g., ADHD-specific rating scales, Generalized Anxiety-specific rating scales, Depression-specific rating scales, etc.). What scales are included in a test set is dependent upon previously published sensitivity (likelihood of a “true positive”) and specificity (likelihood of a “true negative”) for a condition at a designated clinical cut-off score. Thus, each individual rating scale or psychometric test is selected to function within a set of tests. Its published sensitivity/specificity statistics are used to weigh its result in context of the other tests in the set, and in this way, no single measurement is relied upon solely to arrive at an indicated possible condition. For example, the platform 100 screens for symptoms associated with various psychiatric conditions. In some examples, the tests available to the screening engine 108 have conservative sensitivity and have specificity information that is based on published studies that evaluated the rating scale in a clinical sample. In an example, the platform 100 may be configured to be able to assess and evaluate more test sets than a person otherwise would have been able. This may result in a more accurate assessment and may be able to remove biases that may result from a person doing such assessments and evaluations. The platform 100 is utilizing sensitive statistical analyses and/or very specific analyses to make the assessment or evaluation. Moreover, the sensitivity of these evaluations may be able to distinguish more accurately between various psychiatric conditions to provide an assessment and/or evaluation that is more accurate and more comprehensive than a clinician or even multiple clinicians.

The platform 100, via the screening engine 108, provides a life factor questionnaire to be completed by the user (block 206). The life factor questionnaire has the user respond to a series of questions that help populate the report 102, to help the healthcare provider understand the user's full history and help make diagnosis and treatment decisions with the contextual information about the user. The life factor questionnaire may include, for example, demographic information, medical history information, family information, and/or relationship information, etc.

Based on the data provided by the user in the screening, targeted tests, and/or life questionnaire, the platform 100, via the testing engine 110, analyzes possible psychiatric conditions of the user (block 208). The testing engine 110 bases its analysis by using several techniques. By screening all individuals for symptoms associated with multiple psychiatric conditions (including many of the most commonly occurring conditions), the platform 100 detects potentially co-occurring symptoms or conditions. Additionally, the platform 100 uses the data received via the life factor questionnaire to facilitate receiving clinical and person-specific information for each individual. The platform 100 further reduces the likelihood of false negatives by requiring slightly fewer symptoms to obtain a positive screen than is required by the DSM-5 diagnostic manual, and then assessing for elevated conditions based on the detected or suspected symptoms uncovered during screening.

The platform 100 uses a statistical approach to obtain results with a high degree of confidence. Specifically, each of the conditions assessed by the testing engine 110 has a corresponding set of condition-specific rating scales (e.g., ADHD-specific rating scales, Generalized Anxiety-specific rating scales, Depression-specific rating scales, etc.). The sets of rating scales are defined in such a way that scoring above the published cut-off score on every rating scale in the set corresponds to a high probability or a high likelihood (e.g., eighty-five percent (85%) or greater) (sometimes referred to as a “target probability”) that the user has the condition assessed by the test set. That is, if an individual achieves a positive screen on each test within a condition-specific test set (e.g., confirmatory tests, targeted tests based on the screening, individual rating scales or psychometric tests such as ADHD-specific rating scales, Generalized Anxiety-specific rating scales, Depression-specific rating scales, etc.), there is an 85% likelihood that they have symptoms consistent with that condition. The sets of rating scales are constructed using peer-reviewed and published cut-off score, and sensitivity (likelihood of a “true positive”) and specificity (likelihood of a “true negative”) data for each rating scale in each test set. Using these validity statistics for each test, the platform 100 calculates the total probability that the individual has the condition. This probability is displayed in various ways. When multiple cut-offs are available (e.g., in published literature), the test sets are based on the clinical sample data or, in some examples, the test sets are based on the most conservative general population sensitivity and specificity statistics for each rating scale and used those statistics in the odds likelihood calculation for the entire test set. These rating scales are commonly used in the fields of psychology, counseling, and social work and medicine, and are either available in the public domain or requisite permissions to use can be acquired. For instance, Table 1 illustrates an example test set for three individual rating scales for anxiety disorder.

TABLE 1
Example Rating Scales for a Test Set that
Assesses Generalized Anxiety Disorder

| | | Min- | |
|---|---|---|---|
| | Cut-Off | imum | Maximum |
| Screener | Score | Score | Score |
| Generalized Anxiety Disorder-7 | 8.0 | 0.0 | 21.0 |
| Hospital Anxiety Depression Scale - | 8.0 | 0.0 | 21.0 |
| Anxiety Subscale | | | |
| Screen for Adult Anxiety Related Disorders - | 12.0 | 0.0 | 26.0 |
| Generalized Anxiety Disorder Subscale | | | |

The platform 100 determines that a condition has a high likelihood of being present when the following applies: (1) the screening tests report a sufficient number of symptoms and severity or frequency of those symptoms to warrant follow up assessment for a specific condition, and that none of the information gathered during screening ruled-out the possible presence of that specific condition, (2) a user's pattern of responses on the individual tests resulted in an 85% or greater likelihood that they have the condition, for entire test set. The testing engine 110 requires detection of positive screens on all the individual tests within a set in order to obtain “high likelihood” for a particular condition. This reduces the likelihood of false positives.

To arrive at a combined probability percentage determination (e.g., probability percentage of a true positive finding) in the event of unanimous agreement amongst the rating scales, a conservative estimate of probability is used by assigning an assumed error rate to a measure through setting its error equal to (1−Sensitivity)+(1−Specificity). Thus, a measure with peer-reviewed published 80% sensitivity and 80% specificity at a certain clinical cut-off score would be assigned a 40% error rate. Then, a probability level of a true positive finding is calculated for each set of tests designated for a specific disorder or condition. The probability level of a true positive finding is calculated by the formula: 1 [maximum possible probability]−(Error %ₜₑₛₜ₁×Error %ₜₑₛₜ₂× . . . Error %ₜₑₛₜ₍ₙ₎). As an example, if a test set available to the screening engine 108 holds three rating scales, each confirmed to have 80% sensitivity and 80% specificity discriminant validity at a certain cut-off score, the test set's combined likelihood of being correct (e.g., probability level of a true positive finding) in the event of unanimous agreement, would be equal to: 1−(0.40×0.40×0.40)=0.937, or 93.7%. Probability is either expressed as a % or a level of evidence to account for possible overstating confidence in any results.

The platform 100 then provides the report 102 (block 210). The report 102 may have one or more parts. The first part may include a comprehensive psychoeducation on the conditions detected or likely detected by the platform geared towards the user and written by psychologists and clinicians in an empathetic and pragmatic tone. The second part may include information a healthcare provider would need in order to make sound decisions about the user's mental health. The report 102 displays impressions of which conditions are present or are likely present based not only on the elevated assessment results of the condition, but also by ruling out the DSM-5's required “related” or “similar” conditions (e.g., the results of the empirically validated test set for each condition that was elevated in the initial screening stage of the assessment), as well as identifying the information that still needs assessment if the platform 100 was not able to determine a result for a given related condition. The second part of the report 102 may include: (a) demographic information, (b) reason for testing (e.g., as collected by the screening test from the user), (c) relevant medical history, (d) evaluation of suicide risk, (e) data on the possible conditions detected, (0 raw or underlying data supporting the possible detected conditions, and/or (g) drug tables for the relevant conditions, etc. In some examples, the data on the possible conditions detected lists each detected condition along with data associated with the symptoms (e.g., tests/assessments with their likelihood rating or level of evidence rating), other diagnostic criteria set forth by the DSM-5, lists of conditions which it “May Be” that have positives & mixed tests, lists of conditions which it “May Be” that are positive screening only, lists conditions which it “May Be” but for which the platform 100 does not assess, and differential diagnostics results. The data on the possible conditions detected may include scores for conditions where further assessment is needed (e.g., conditions the platform 100 detected with a low degree of confidence and/or conditions that were ruled out.) Additionally, the data on the possible conditions detected includes results/scores, cutoffs, and/or score ranges per test.

FIG. 3 illustrates a system architecture 300 that may be used to implement the present disclosure. In particular, the computing device 104 (e.g., user device, display device, etc.) may be in communication with a host computing device 304, comprising a memory 302 and a processor 303, by way of a communication framework 306 such as the interne, network, or cloud as is generally known in the art. The host computing device 304 and communication framework 306 of the system architecture 300 may also include a web server that provides access to the platform 100 (e.g., a psychological assessment web application). Upon accessing the platform 100, the user may be prompted to input user provided information 320 at the computing device 104. For example, the user may be prompted to provide information to create a profile, and the user may also be prompted to provide answers for the screening tests (e.g., a multiple-tiered process), confirmatory tests (e.g., targeted tests based on the screening, individual rating scales, psychometric tests, etc.), and questionnaires (e.g., life factor questionnaires). The user provided information 320 may include, by way of a non-limiting example, the user's name, email, age, and other data required to generate a psychological assessment report (e.g., report 102).

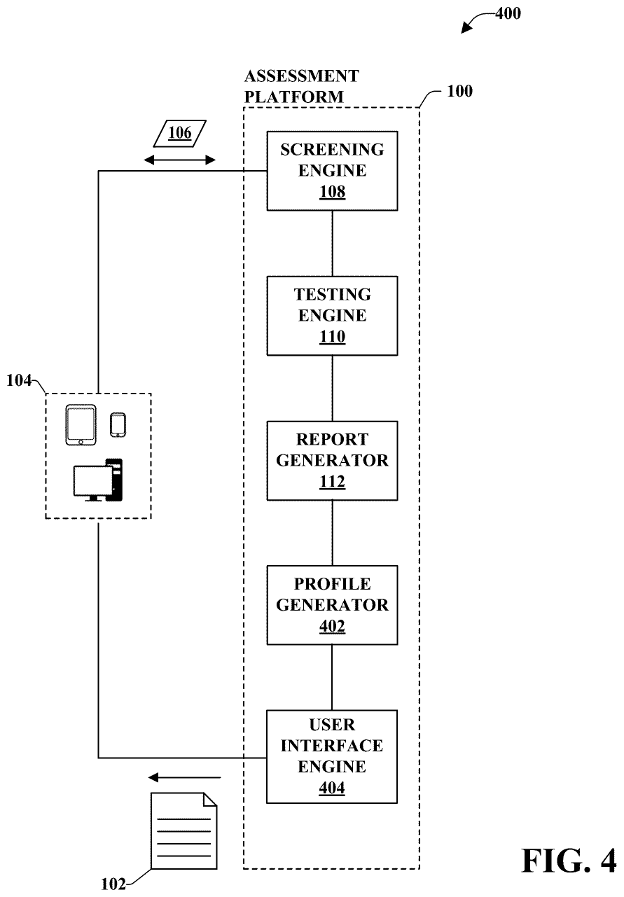

FIG. 4 illustrates an example system 400, which is similar to system 10, and further comprising a profile generator 402 and a user interface engine 404. The profile generator 402 can prompt a user to provide information such as name and age to create a user profile. The user interface engine 404 can display the user profile onto the computing device 104 for the user to confirm whether the information displayed is correct. The user interface engine 404 can also display additional questions needed to generate a user profile. In addition, the user interface engine 404 can also display the questions provided by the screening engine 108 and the report 102 generated by the report generator 112.

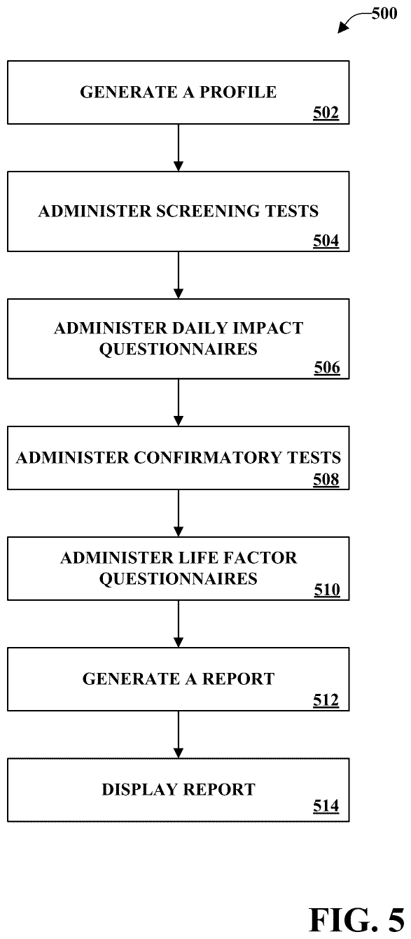

FIG. 5 is an example flowchart of a method 500 in accordance with the embodiments disclosed herein. Aspects of the embodiments disclosed herein provides a digital psychological assessment tool that selects, administers tests and questionnaires, generates reports (e.g., report 102), and provides mental health-promoting recommendations based on a user's answers to the tests and questionnaires. The tests/questionnaires begin with preliminary symptom checklists (e.g., screening tests) and is followed by sets of peer-reviewed and published self-report psychiatric screening scales (e.g., confirmatory tests) with established validity and reliability. For example, the patient health questionnaire-9 (PHQ-9) is a screening scale (e.g., a confirmatory test, rating scale, etc.) that assesses the degree of severity for depression.

The method 500 may comprise of generating (e.g., by a profile generator 402 of system 400 which is similar to the system 10; see e.g., FIG. 4) a profile for a user based on a user's input, via the computing device 104, upon registering on a website and providing characteristics such as, but not limited to, name, email, and age (block 502). The user may be prompted by the screening engine 108 to provide the characteristics and any additional information needed to generate a profile.

The method 500 may further comprise administering (e.g., by the screening engine 108) a set of one or more screening tests (block 504), which may prompt the user to answer a checklist of common signs and symptoms of more than 50 psychiatric conditions to provide the system 400 with a list of conditions (e.g., hypothesized conditions) for which it will administer a set of well-researched and well-known rating scales (e.g., confirmatory tests). The symptom checklist has a one-to-one alignment with the symptoms enumerated in the DSM-5, so it is multiple-tiered, and the symptom checklist prompts the user to endorse the presence or absence of each symptom for a given condition.

By way of non-limiting example, the screening tests may have three tiers. Tier 1 may include questions regarding broadly described symptoms that are required per the DSM-5 for certain categories of DSM-5 disorders. An example Tier 1 question may include: Do you experience excessive worrying, panic, or fearfulness? Another example Tier 1 question may include: You worry a lot, more than you would like, or more than you think is typical for a given situation. These questions may have multiple-choice answers that include: Yes, No, or I'm Unsure.

Tier 2 may include questions regarding symptoms whose presence is required for a certain DSM-5 disorders to be present. An example Tier 2 question may include: Has your anxiety or worrying been bothering you on more days than not, for approximately the last six months or longer?

Tier 3 may include questions regarding symptoms of which the presence of a certain amount is required for certain DSM-5 disorders to be present. Example Tier 3 questions may include: (i) You feel muscle tension; (ii) You are restless, keyed up, or on edge; (iii) You are easily fatigued; etc.

The method 500 may further comprise administering a set of one or more daily impact questions (block 506). The daily impact questions may include questions about how much the symptoms impact the user's daily life. A user may have symptoms of various mental health conditions, so in order to determine whether a user meets the level of clinical classification, it may be useful to understand the ways these symptoms impact the user's daily life. The daily impact questions may ask what life looks like for the user and how these symptoms affect the user. The response from these questions may facilitate in generating a list of hypothesized conditions for further targeted testing.

If, based on a user's characteristics and symptom endorsements the presence of one or more DSM-5 disorders is possible (e.g., meaning that disorder-specific Tier 3 symptoms were endorsed and at least one less than the DSMS-required volume of disorder-specific Tier 3 symptoms were endorsed), the method 500 may further comprise administering (e.g., by the screening engine 108) a set of one to four established and well known self-report survey-type rating scales (e.g., confirmatory tests) validated for use in screening for possible disorders (block 508). It is appreciated that the set may have less than two or more than four established and well known self-report survey-type rating scales.

The method 500 may further comprise administering a set of one or more life factor questionnaires (block 510). The life factor questionnaires may include questions about a user's life that may help a physician (e.g., healthcare provider) tailor treatments for the user. A user may have a unique history that contributes to how the user is feeling, so these life factor questions may facilitate in constructing (e.g., by the report generator 112) a personalized report (e.g., report 102) (block 512). The personalized report may be provided to the user by displaying (e.g., by the user interface engine 404) the report onto computing device 104 (block 514).

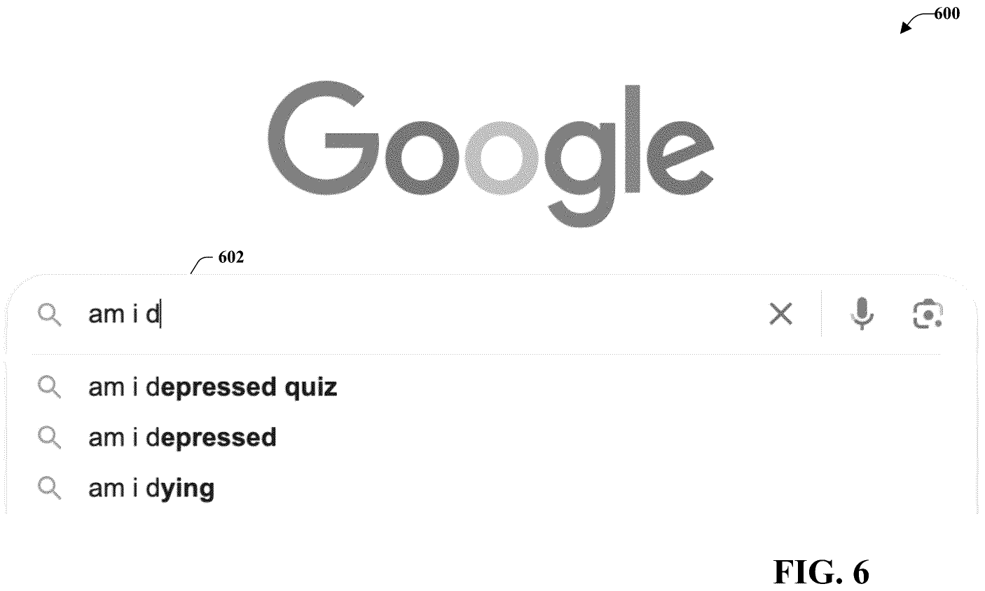

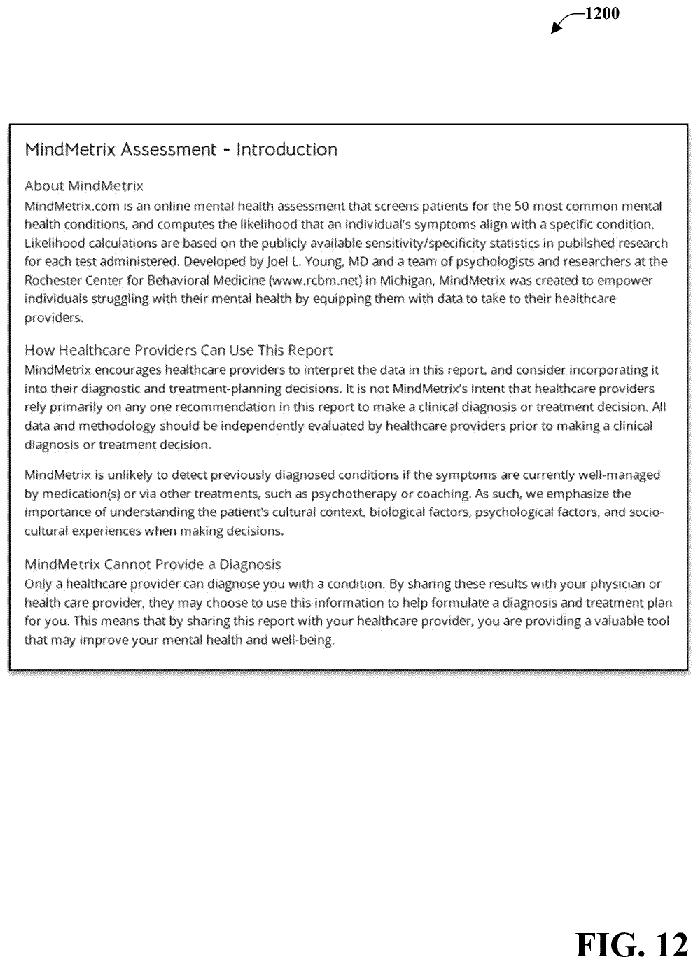

FIGS. 6-18 illustrate an example user journey of a fictitious user, Jim Nash. Jim has not felt right lately. He gets anxious leaving home and finds it difficult to breathe in certain situations. He went to his primary care physician (PCP), who worked him up for asthma and allergies but did not find anything remarkable. Jim's symptoms remain, and he is left wanting something credible that might shed light on his symptoms and offer a path forward, so searches the interne. FIG. 6 illustrates Jim using a search engine 600 to search for more information on his condition in search bar 602.

FIG. 7 illustrates that Jim discovers the platform 100 and visits the website, and through a series of displays 700, Jim answers screening tests. He likes that he can see whether his symptoms might match a wide range of conditions and that he can take data to a healthcare provider. Display 702 informs Jim that for the first part of the assessment, it is important to get an understanding of the types of mental health symptoms he has been experiencing, that he will be presented with a series of 19 symptom areas, and that his responses to each question in this section will determine the questions asked in the rest of the assessment. So Jim creates an account. He answers a DSM-5-based questionnaire about his symptoms spanning a wide range of mental health conditions. It is appreciated that the number of symptoms areas (or conditions) as well as the number of questions or type of questions, and the like, described here and throughout may change, especially as new understandings and discoveries are made.

As Jim begin his journey, displays 704 and 706 ask Jim a series of questions. The display 704 prompts Jim to answer yes, no, or “I'm unsure” whether he has ever experienced sadness, depression, or numbness for at least several days at a time. Depending on Jim's response from the display 704, the display 706 may inform Jim that a deeper understanding into his problems with attention is needed and prompts him to answer more questions. The display 706 may ask Jim to respond yes or no whether he is losing focus even during activities that he enjoys and whether he is losing track of what others say to him.

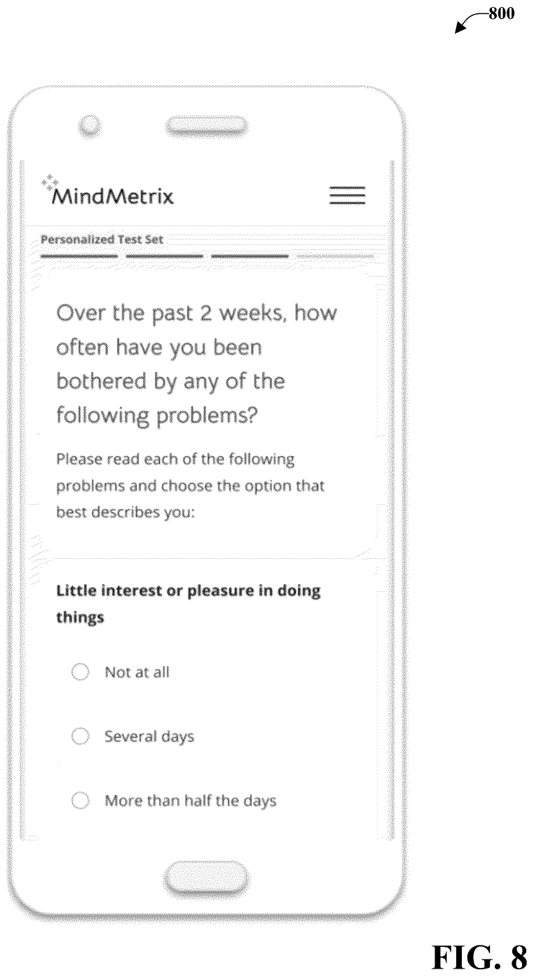

FIG. 8 illustrates display 800 prompting Jim to answer validated rating scales (e.g., confirmatory tests). Jim uses the questionnaire to self-report his symptoms. He reports elevated symptoms across 7 different conditions. He is presented with a set of 3 to 4 validated rating scales for each of the 7 conditions. For example, the display 800 may ask Jim to read a series of problems and choose the answer that best describe how often any of those problems have been bothering him over the past 2 weeks. It is appreciated that additional types of questions may be asked. For example, as previously described, daily impact and life factor questions may asked to generate a more personalized report of a user.

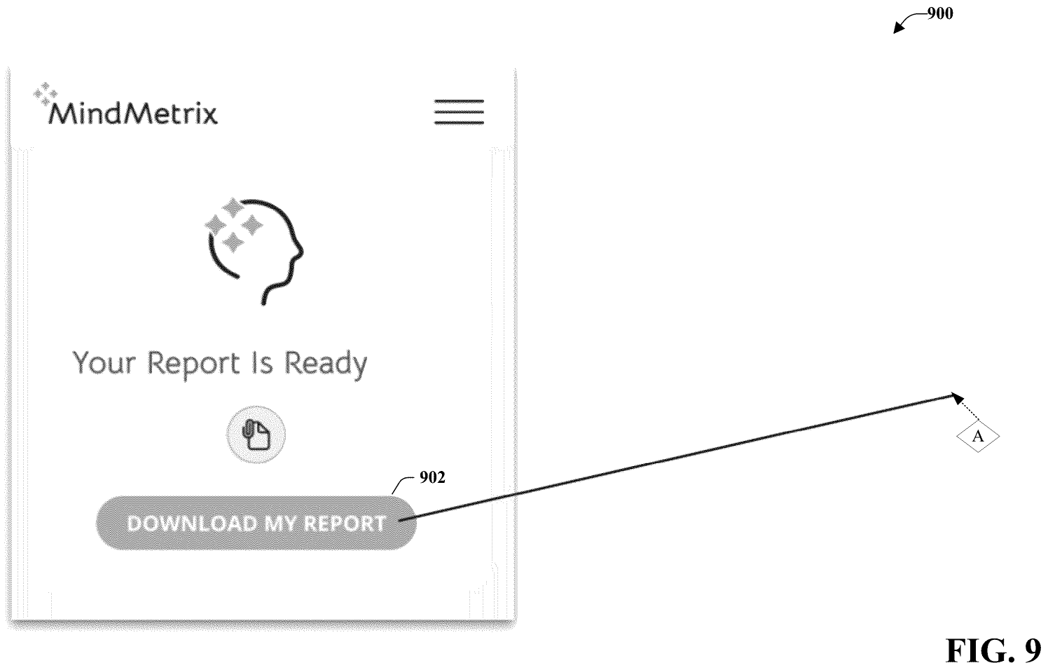

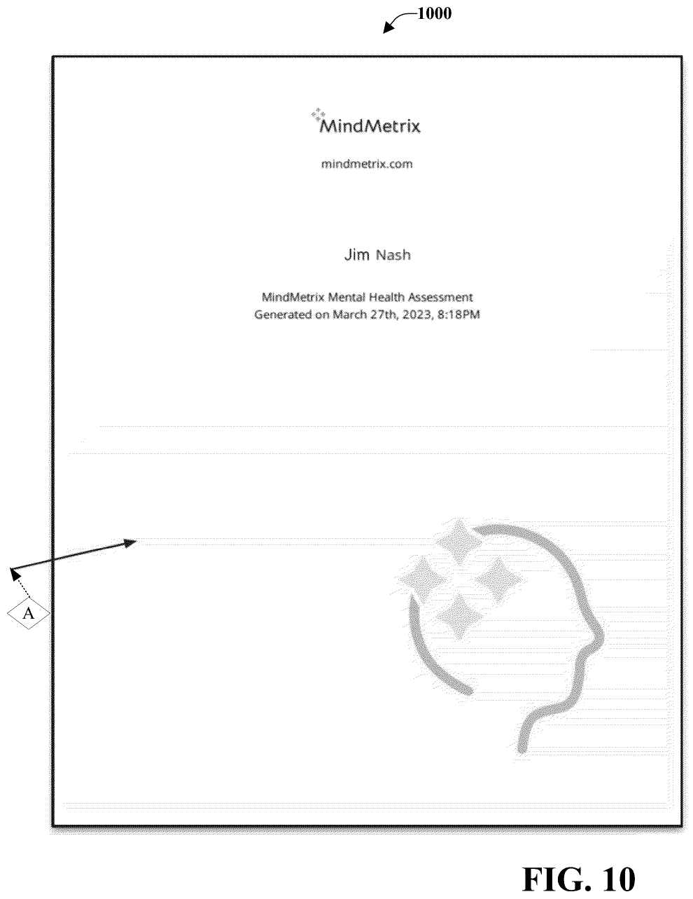

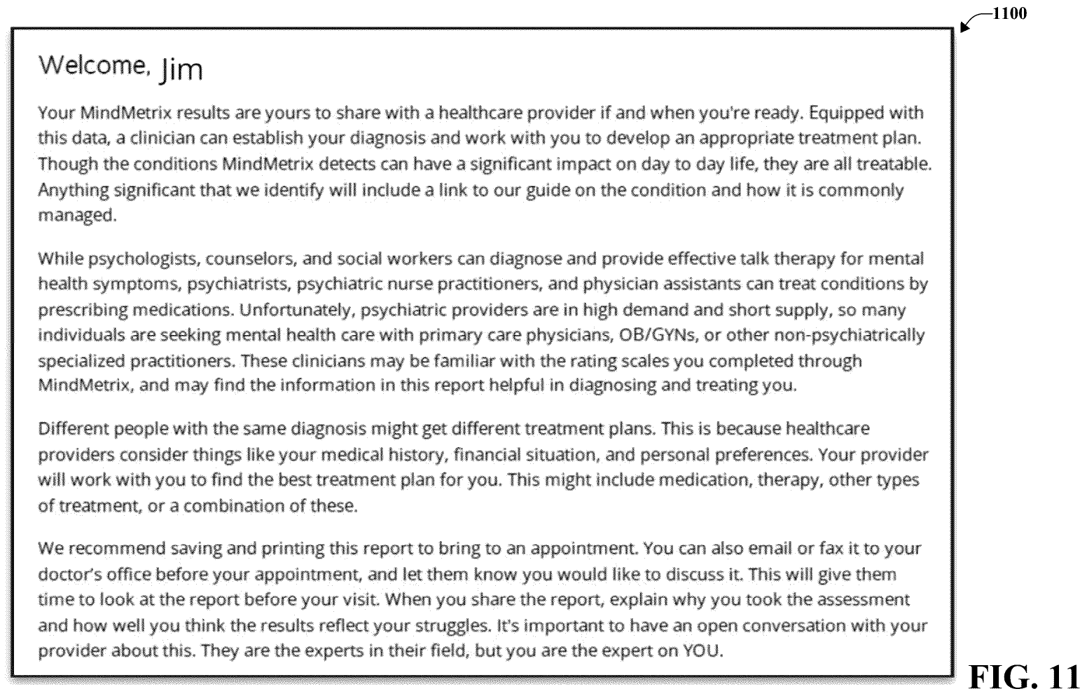

Jim submits his response, and FIG. 9 illustrates Jim is able to download a report immediately after taking the assessment. Jim clicks on the download-my-report button 902, and he is presented with report 1000 (e.g., report 102) from The platform 100, as illustrated in FIG. 10. The report 1000 is generated by the report generator 112 and displayed for the user on the computing device 104 via the user interface engine 404. The report 1000 is shown through the computing device 104 and includes a series of displays 1100 through 1800, as illustrated in FIGS. 11-18.

FIG. 11 illustrates an example welcome message reminding the user Jim that he can share the results of the report 1000 with his healthcare provider. Equipped with the data from the report 1000, a clinician can establish Jim's diagnosis and work with him to develop an appropriate treatment plan. As the report 1000 can be read by both Jim and his healthcare provider, both Jim and his healthcare provider are informed that though the conditions detected for Jim can have a significant impact on day to day life, they are all treatable. It is recommended that Jim saves and prints his report to bring to his appointment. He can also email or fax his report to his doctor. While psychologists, counselors, and social workers can diagnose and provide effective talk therapy for mental health symptoms, psychiatrists, psychiatric nurse practitioners, and physician assistants can treat conditions by prescribing medications. Unfortunately, psychiatric providers are in high demand and short supply, so many individuals are seeking mental health care with primary care physicians, obstetricians/gynecologists (OB/GYNs), or other non-psychiatrically specialized practitioners. These non-psychiatrically specialized practitioners may be familiar with the rating scales in the report 1000 and may find the information in the report 1000 helpful in diagnosing and treating Jim.

A display 1200 is provided with an introduction to the platform 100 assessment (e.g., assessment platform 100 via system 10 or system 400). The introduction states that the platform 100. com is an online mental health assessment that screens patients for the 50 most common mental health conditions, and computes the likelihood that an individual's symptoms align with a specific condition. Likelihood calculations are based on the publicly available sensitivity/specificity statistics in published research for each test administered. The platform 100 was created to empower individuals struggling with their mental health by equipping them with data to take to their healthcare providers. The platform 100 encourages healthcare providers to interpret the data in this report, and consider incorporating it into their diagnostic and treatment-planning decisions. It is not the platform 100's intent that healthcare providers rely primarily on any one recommendation in this report to make a clinical diagnosis or treatment decision. All data and methodology should be independently evaluated by healthcare providers prior to making a clinical diagnosis or treatment decision. The platform 100 is unlikely to detect previously diagnosed conditions if the symptoms are currently well managed by medications(s) or via other treatments, such as psychotherapy or coaching. As such, it is emphasized that the importance of understanding the patient's cultural context, biological factors, psychological factors, and sociocultural experiences when make decisions. It is appreciated that these factors may be asked in the daily impact questionnaires or life factor questionnaires or any number of additional questionnaires may be added. Jim is also reminded that only a healthcare provider can diagnose him with a condition. By sharing these results from the report 1000 with his physician or healthcare provider, they can choose to use this information to help formulate a diagnosis and treatment plan for him. By sharing this report to his healthcare provider, he is providing a valuable tool that may improve his mental health and well-being.

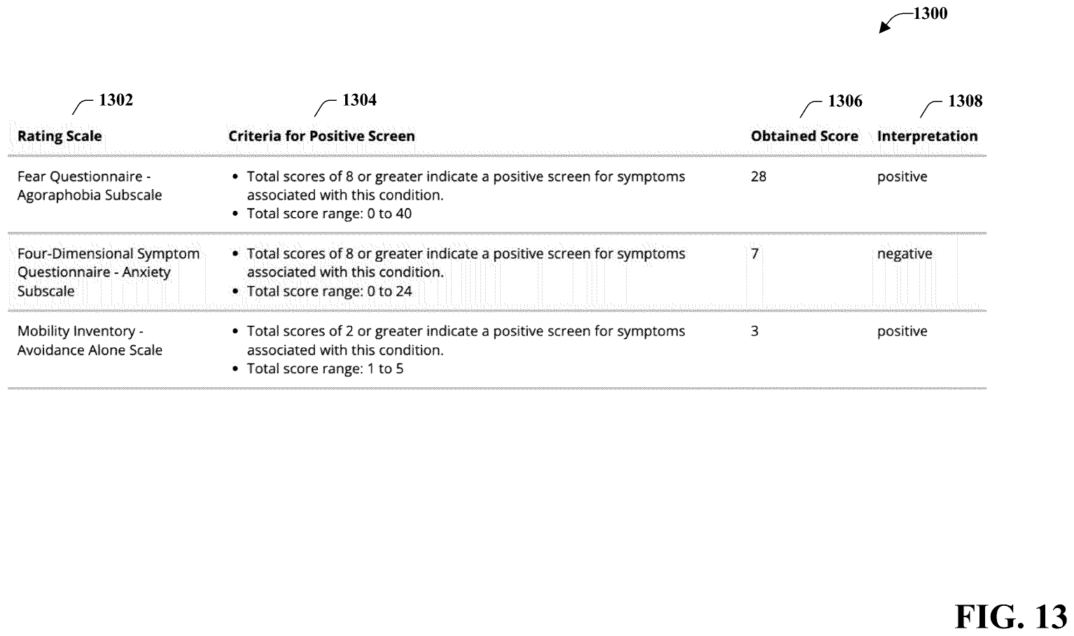

FIG. 13 illustrates a display 1300 comprising a rating scale 1302, criteria for positive screen 1304, obtained score 1306, and interpretation 1308. Through the display 1300, Jim learns about the rating scales he has taken and their results. For example, the agoraphobia subscale (1302) requires (1304) total scores of 8 or greater to indicate a positive screen for symptoms associated with this condition, and the total score range is 0 to 40. Jim obtained a score (1306) of 28 for agoraphobia which is interpreted (1308) as a positive screen. The anxiety subscale (1302) requires (1304) total scores of 8 or greater to indicate a positive screen for symptoms associated with this condition, and the total score range is 0 to 24. Jim obtained a score (1306) of 7 for anxiety which is interpreted (1308) as a negative screen. The avoidance alone scale (1302) requires (1304) total scores of 2 or greater to indicate a positive screen for symptoms associated with this condition, and the total score range is 1 to 5. Jim obtained a score (1306) of 3 for avoidance alone which is interpreted (1308) as a positive score.

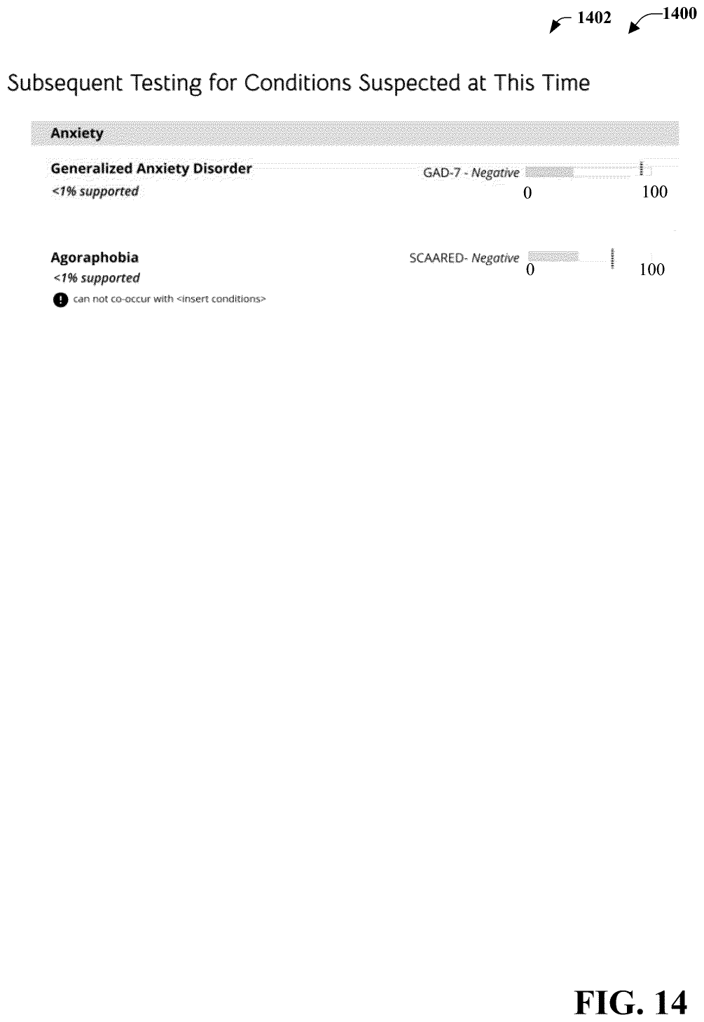

FIG. 14 also illustrates a display 1400 showing Jim a summary of his elevated symptom profiles, e.g., subsequent testing for conditions suspected at this time. The display 1400 shows Jim a list 1402 of possible conditions and results of each administered set of ratings. The % helps him understand which conditions his symptoms most closely align with. Bayes' formula for independent conditional probability is used to help the reader incorporate published sensitivity/specificity information across each set of scales. The display 1400 shows elevated symptom profiles for anxiety, wherein the General Anxiety Disorder-7 (GAD-7) score is negative and the Screen for Adult Anxiety Related Disorders (SCAARED) score is negative. It is appreciated that additional elevated symptom profiles may be included.

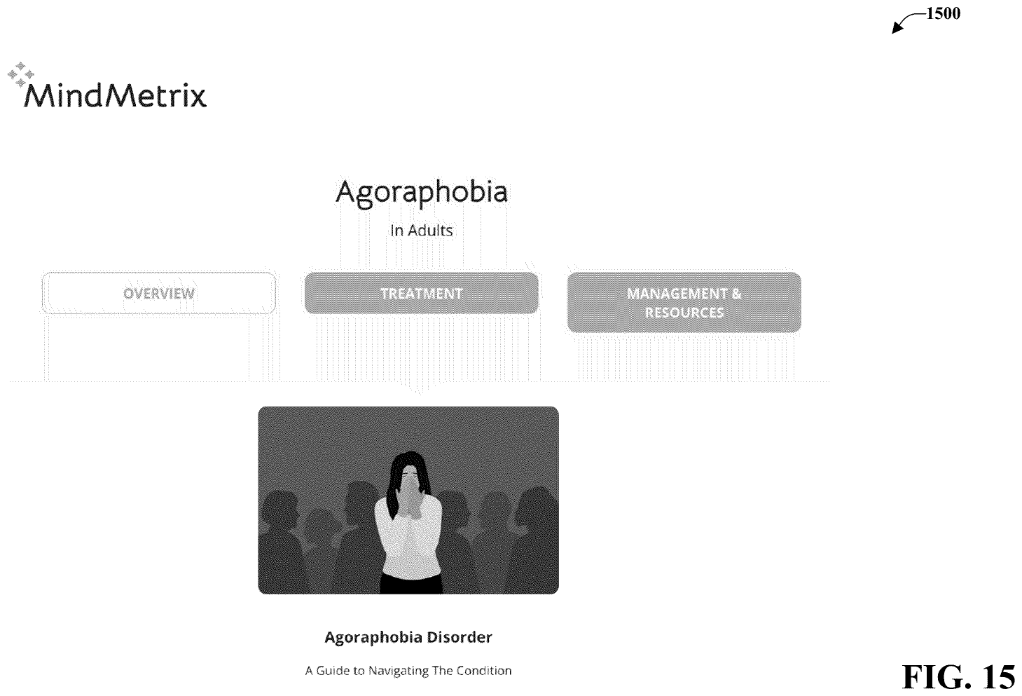

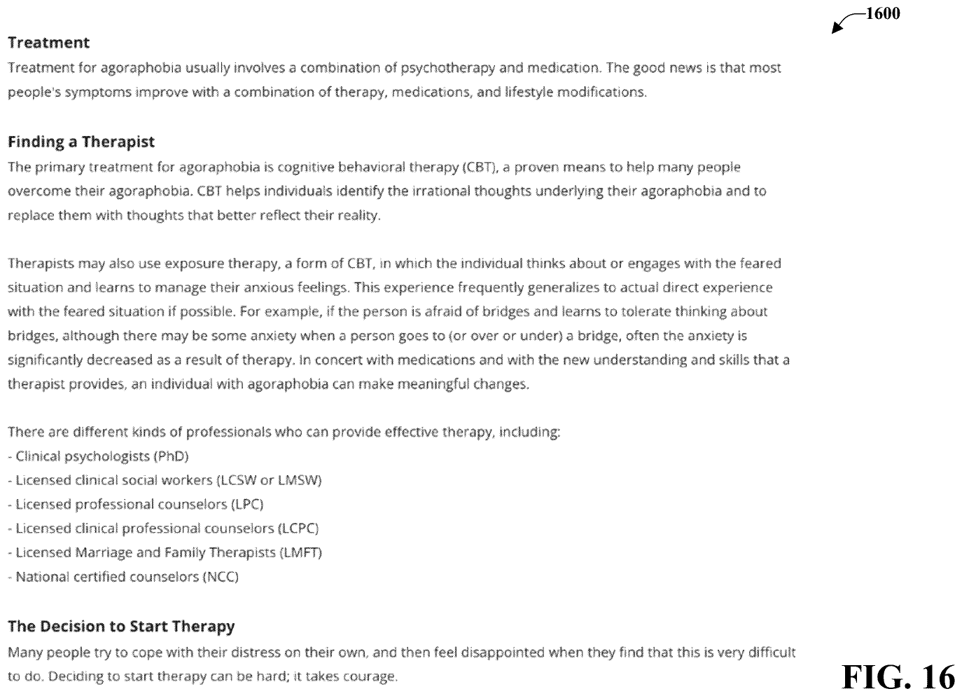

FIG. 15 illustrates a display 1500 for agoraphobia disorder in adults with options to navigate for more information such as an overview of the disorder, treatment options, or management and resources for this disorder. FIG. 16 illustrates display 1600 showing an overview of the agoraphobia disorder. The display 1600 provides an overview of treatment options and the different kinds of professionals that can provide effective therapy for this condition. The display 1600 provides that treatments for agoraphobia usually involve a combination of psychotherapy and medication. Most people's symptoms improve with a combination of therapy, medications, and lifestyle modifications. The primary treatment for agoraphobia is cognitive behavioral therapy (CBT), a proven means to help may people overcome their agoraphobia. CBT helps individuals identify the irrational thoughts underlying their agoraphobia and to replace them with thoughts that better reflect their reality. Therapists may also use exposure therapy, a form of CBT, in which the individual thinks about or engages with the feared situation and learns to manage their anxious feelings. In concert with medications and with the new understanding and skills that a therapist provides, an individual with agoraphobia can make meaningful changes. There are different kinds of professionals who can provide effective therapy, including: clinical psychologists (PhD), licensed clinical social workers (LCSW or LMSW), licensed professional counselors (LPC), licensed clinical professional counselors (LCPC), licensed marriage and family therapists (LMFT), or national certified counselors (NCC).

After reading the agoraphobia guide, Jim finally felt “seen” and for the first time, he decides to make an appointment with a therapist who specializes in phobias. Jim also makes another appointment with his PCP. Both providers are informed of the DSM-5 required criteria for a diagnosis of agoraphobia and can see common treatment for it.

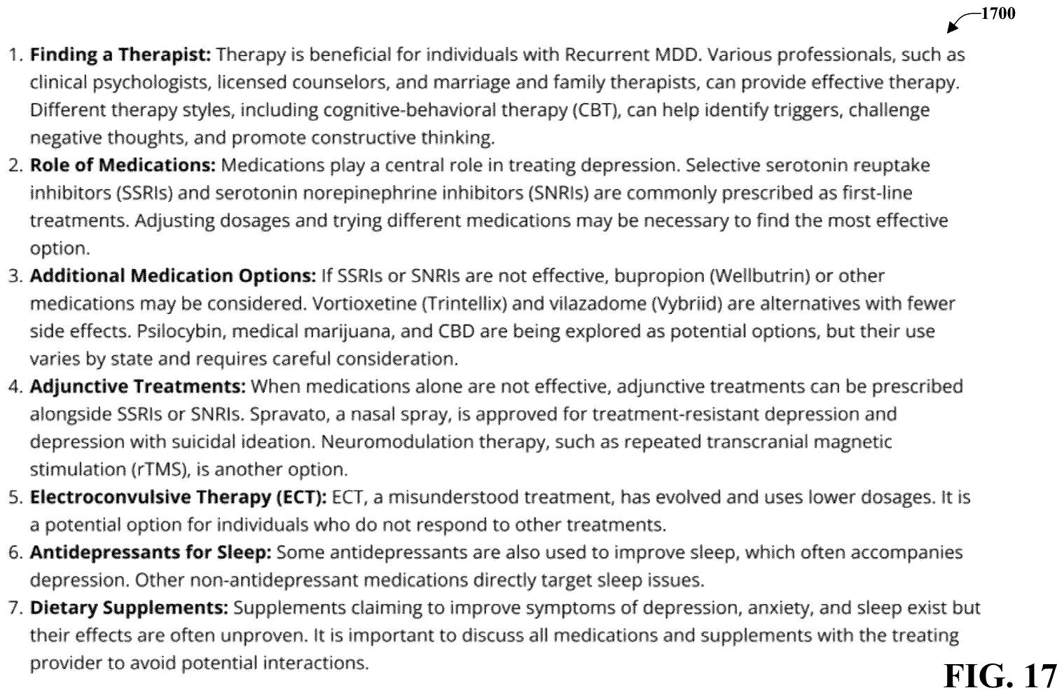

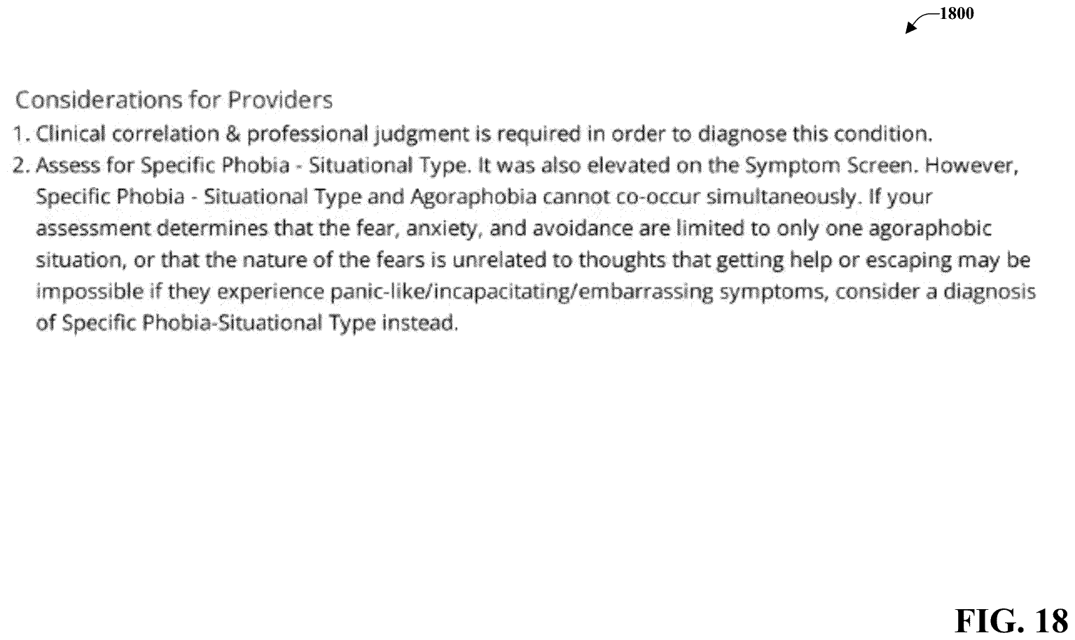

FIG. 17 illustrates display 1700 providing treatment options such as, but not limited to, finding a therapist, medications, additional medication options, adjunctive treatments, electroconvulsive therapy (ECT), antidepressants for sleep, and dietary supplements. FIG. 18 illustrates display 1800 for considerations for healthcare providers. The display 1800 provides a reminder that clinical correlation and professional judgment is required in order to diagnose this condition. The display 1800 also provides additional considerations for healthcare providers when diagnosing this condition using the report 1000.

Although the embodiments of the present invention have been illustrated in the accompanying drawings and described in the foregoing detailed description, it is to be understood that the present disclosure is not to be limited to just the embodiments disclosed, but that the disclosure described herein is capable of numerous rearrangements, modifications and substitutions without departing from the scope of the claims hereafter. The terms “includes,” “including,” and “include” are inclusive and have the same scope as “comprises,” “comprising,” and “comprise” respectively. The claims as follows are intended to include all modifications and alterations insofar as they come within the scope of the claims or the equivalent thereof.

Having thus described the invention, the following is claimed:

1. A platform to provide digital assessments of mental health comprising:
a screening engine that provides screening tests, confirmatory tests, or questionnaires to a user and receives input from the user via a display device;
a testing engine that assesses information gained from the screening tests, confirmatory tests, or questionnaires against a set of condition-specific rating scales to determine diagnostic specifiers that are indicative of existence, non-existence, or likelihood of one or more mental health conditions; and
a report generator that generates a report based on the diagnostic specifiers.

2. The platform of claim 1 further comprising a profile generator that generates a profile for the user based on a user's input upon registering on a website and providing characteristics such as, but not limited to, name, email, and age.

3. The platform of claim 1 wherein the screening engine screens the user for symptoms associated with psychiatric conditions through a series of questions based on a Diagnostic and Statistical Manual of Mental Disorders, Fifth Edition (DSM-5).

4. The platform of claim 1 wherein the report includes a user's response to life factor questions that are further provided by the screening engine.

5. A system comprising:
a host computing device that includes a web server and an application server; and
a user device in communication with the host computing device by way of a communication framework to provide a user access to an assessment platform via the web server and the application server;
wherein the assessment platform comprises:
a screening engine that provides screening tests, confirmatory tests, or questionnaires to a user and receives input from the user via a display device;
a testing engine that assesses information gained from the screening tests, confirmatory tests, or questionnaires against a set of condition-specific rating scales to determine diagnostic specifiers that are indicative of existence, non-existence, or likelihood of one or more mental health conditions; and
a report generator that generates a report based on the diagnostic specifiers.

6. The system of claim 5 wherein the assessment platform further comprising a profile generator that generates a profile for the user based on a user's input upon registering on a website and providing characteristics such as, but not limited to, name, email, and age.

7. The system of claim 5 wherein the screening engine screens the user for symptoms associated with psychiatric conditions through a series of questions based on a Diagnostic and Statistical Manual of Mental Disorders, Fifth Edition (DSM-5).

8. The system of claim 5 wherein the assessment platform, via the testing engine, determines that one or more mental health conditions have been supported or not when a user's pattern of responses on individual tests resulted in a majority of positive versus scores for a specific condition.

9. The system of claim 5 wherein the report comprises a first part that includes a comprehensive psychoeducation and a second part that includes information a healthcare provider would need in order to make sound decisions about a user's mental health.

10. The system of claim 9 wherein the second part comprises: (a) demographic information, (b) reason for testing, (c) relevant medical history, (d) evaluation of suicide risk, (e) data on possible conditions detected, (f) raw or underlying data supporting possible conditions detected, or (g) drug tables for relevant conditions.

11. A method comprising:
administering screening tests, confirmatory tests, or questionnaires to a user and receiving input from the user via a user device;
assessing information gained from the screening tests, confirmatory tests, or questionnaires against sets of condition-specific rating scales to determine diagnostic requirements that are indicative of an existence, non-existence, or likelihood of a mental health condition; and
generating a report based on the diagnostic requirements.

12. The method of claim 11 wherein the screening tests prompt the user to answer a checklist of common signs and symptoms of more than 50 psychiatric conditions to provide a list of hypothesized conditions for further testing.

13. The method of claim 12 wherein the checklist of common sign and symptoms has a one-to-one alignment with symptoms enumerated in a Diagnostic and Statistical Manual of Mental Disorders, Fifth Edition (DSM-5), and the checklist of common sign and symptoms prompts the user to endorse whether the symptoms for a given condition is presence or absence.

14. The method of claim 13 wherein the screening tests comprises three tiers, and wherein:
Tier 1 comprises questions regarding broadly described symptoms that are required per the DSM-5 for certain categories of DSM-5 disorders;
Tier 2 comprises questions regarding symptoms whose presence is required for certain DSM-5 disorders to be present; and
Tier 3 comprises questions regarding the symptoms of which the presence of a certain amount or timeframe is required for the certain DSM-5 disorders to be present.

15. The method of claim 11 further comprising administering a set of one or more daily impact questions about how symptoms impact a user's daily life.

16. The method of claim 14 wherein if, based on a user's characteristics and symptom endorsements the presence of one or more DSM-5 disorders is possible, the method further comprises:
administering a set of one to four established and well -known self-report survey-type rating scales validated and peer-reviewed for use in screening for possible disorders.

17. The method of claim 16 wherein whether the presence of one or more DSM-5 disorders is possible means that disorder-specific Tier 3 symptoms were endorsed and at least one less than a DSMS-required volume of the disorder-specific Tier 3 symptoms were endorsed.

18. The method of claim 11 further comprising administering a set of one or more life factor questionnaires that comprises questions about a user's life that may help a healthcare provider tailor treatments for the user.

19. The method of claim 11 wherein based on sensitivity and specificity data of the sets of condition-specific rating scales, a probability level of a true positive finding or level of evidence is calculated by a formula: 1 [maximum possible probability]−(Error %ₜₑₛₜ₁×Error %ₜₑₛₜ₂× . . . Error %ₜₑₛₜ₍ₙ₎), wherein
a conservative estimate of probability is used by assigning an assumed error rate to a measure through setting an error value equal to: (1−Sensitivity)+(1−Specificity), and wherein
the conservative estimate of probability may be presented as a % for each condition or as a level of evidence such as strong, moderate, or mild.

11. The method of claim 11 further comprising displaying the report on a user device.
